(19) United States
(12) Patent Application Publication
(10) Pub. No.: US 2017/0360595 A1
(43) Pub. Date: Dec. 21, 2017

(54) Intraoral Orthosis for Mandibular Advancement for Snoring and Obstructive Sleep Apnea

(71) Applicant: Jacob Smith, Strathfield (AU)

(72) Inventor: Jacob Smith, Strathfield (AU)

(21) Appl. No.: 15/597,802

(22) Filed: May 17, 2017

(30) Foreign Application Priority Data
May 18, 2016 (AU) 2016901853

(51) Int. Cl. A61F 5/56

(52) U.S. Cl. CPC A61F 5/566

(57) ABSTRACT

Provided herein is an intraoral orthosis to remedy snoring and/or sleep apnoea via a mandibular advancing action during sleep. Maxilla and mandibular retainers fit the teeth and are coupled with a pair of side retention straps to advance the lower jaw to prevent narrowing of the pharynx. Abutting pairs of planar occlusal faces on the maxilla and mandibular retainers allow lateral and anterioral sliding against each other. A ball engagement couples the retainers to the retention straps at opposing distal ends.

### CROSS-REFERENCE TO RELATED APPLICATIONS

This non-provisional application claims benefit of priority under 35 U.S.C. §119(a) of Australian provisional application number AU 2016901853, filed May 18, 2016, the entirety of which is hereby incorporated by reference

### BACKGROUND OF THE INVENTION

### Field of the Invention

The present invention relates to intraoral orthosis for mandibular advancement for snoring and obstructive sleep apnoea.

### Description of the Related Art

Snoring and obstructive sleep apnoea is caused by the relaxation of the throat muscles during sleep causing a narrowing of the pharynx. This restriction causes a venturi-type effect which vibrates the soft palate and uvula causing snoring. In extreme cases, breathing may be obstructed completely for several seconds called sleep apnoea, resulting in the periodic and noisy resumption of breathing.

Such restriction may be addressed by the advancement of the lower jaw and many intraoral orthoses exist for such purpose. Such known intraoral orthoses may comprise maxilla and mandibular teeth fitting retainers operably coupled together by pivoting lateral retention straps such that the lower jaw is held in an advanced position with respect to the upper jaw.

However, such known intraoral orthoses comprises limitations in restricting jaw movement. Patients that have sleep apnoea often have issues with clenching and grinding at night. The amount of force that can be generated at night with clenching and grinding can be significant and is often substantially more than is produced whilst awake. When a patient that is a heavy bruxer uses an orthoses that does not allow for movement, it is possible to damage the appliance. In some cases, holding the mandible in an unfavourable position can lead to the TMJ dysfunction. For patients with an already damaged or pathologic TMJ, restricting movement may lead to greater damage.

Furthermore, not all orthoses eliminate sleep apnoea entirely, and even while wearing an orthosis, short periods of apnoea may occur wherein the patient will attempt to move the mandible forward to alleviate the airway. However, current orthoses restrict such movement which may reduce efficacy of treatment and furthermore may contribute to TMJ dysfunction as the orthoses resists such forward motion.

As such, it is an object of the present invention to provide an intraoral mandibular advancing orthosis that provides for greater freedom of jaw movement that substantially ameliorates disadvantages of known orthosis, or at least provides an alternative thereto. It is to be understood that, if any prior art information is referred to herein, such reference does not constitute an admission that the information forms part of the common general knowledge in the art.

### SUMMARY OF THE INVENTION

There is provided herein a mandibular advancing intraoral orthosis for addressing snoring and/or sleep apnoea comprising teeth fitting maxilla and mandibular retainers operably coupled together with a pair of lateral retention straps to hold the lower jaw in an advanced position with respect to the upper jaw while sleeping so as to prevent narrowing of the pharynx.

The present orthosis is configured to provide lateral, and, in embodiments, anterior movement tolerance of the mandibular retainer with respect to the maxilla retainer during function.

Specifically, the maxilla and mandibular retainers comprise abutting planar occlusal faces which allow the retainers to slide against each other, both laterally and anteriorly.

Furthermore, the specific coupling of the retention straps to the retainers confers freedom for such movement and prevents breakage and/or dislocation of the couplings as the case may be for pivotal couplings of known orthoses.

As such, with the foregoing in mind, in accordance with one aspect, there is provided an intraoral orthosis comprising: a maxilla retainer; a mandibular retainer; a pair of lateral retention straps coupling the maxilla retainer to the mandibular retainer, each retention strap spanning between an canine region of the maxilla retainer to a posterior molar region of the mandibular retainer and each retention strap comprising an elongate portion wherein: the maxilla and mandibular retainers comprise respectively abutting pairs of planar maxilla and mandibular retainer occlusal faces facilitating at least lateral sliding of the mandibular retainer against the maxilla retainer; and the retainers are coupled to the retention straps by couplings at opposing distal ends thereof wherein each coupling comprises: a lateral retainer buttress comprising an opening to a semi-spherical socket therein; a medial retention strap ball engagement, the ball engagement comprising: a medial rod extending substantially perpendicularly to the elongate portion and terminating in a medial semispherical ball head, the ball head configured for insertion through the buttress opening for engagement and rotatable coupling within the semispherical socket.

The socket design may allow for sufficient flexibility in using a polyamide material to favour insertion and removal of the ball head whilst providing sufficient retention such that the ball head is not displaced during function.

The rod may comprise an exterior diameter less than that of an interior diameter of the opening.

The rod may comprise a rod length and a rod outer diameter and wherein the buttress opening may comprise an opening inner diameter and wherein the buttress may comprise an buttress outer diameter and wherein the rod length, rod outer diameter, opening inner diameter and buttress outer diameter configured such that an inner face of the elongate portion avoids pinching of respective anterior or posterior edges of the buttress through a lateral range of motion of the mandibular retainer with respect to the maxilla retainer.

The range of motion may be approximately −15° to 15° with respect to the sagittal plane.

The retainer buttress may be generally cylindrical.

The retainer buttress may be integrally formed with a respective retainer.

The elongate portion may be angled at a pair of distal junctures such that, in a side elevation view, an anterior distal end may be raised with respect to a posterior distal end.

At least one of the pairs of planar maxilla and mandibular retainer occlusal faces may be raised so as to provide an occlusal offset between the maxilla retainer and the mandibular retainer.

Each retention strap may comprise a linkage allowing shortening of the retention strap under compression.

The linkage may comprise the elongate portion being bifurcated into two constituent distal elongate portions and wherein adjoining edges of the two distal elongate portions may comprise apertures therein through which a link may be retained.

The maxilla and mandibular retainers may comprise a patient specific teeth fitting inner profile.

The maxilla and mandibular retainers are manufactured from a custom fabrication process using patient specific intraoral scanning data or scanning of models of the patient's dentition.

The length of the retention straps may be custom fabricated in accordance with the intraoral scanning data and a desirous retainer offset along the occlusal plane.

The side retention straps are non-elastic and may be manufactured from type 12 polyamide

The side retention straps may comprise metal such as titanium, cobalt chrome and stainless steel.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a front view of a mandibular advancing intraoral orthosis in accordance with an embodiment.

FIG. 2 shows a side view of the orthosis showing a retention strap and connections therefor in accordance with an embodiment.

FIG. 3 shows an orthosis illustrating the disconnection of a side retention strap.

FIG. 4 shows a front view of the orthosis with the retention straps having been removed.

FIG. 5 shows a side view of the orthosis illustrating one of the retention straps.

FIG. 6 shows a side view of the orthosis with the side retention strap having been removed.

FIG. 7 and FIG. 8 show a top and bottom views of the maxilla and mandibular retainers from the occlusal plane illustrating the abutting planar occlusal faces for facilitating lateral motion in accordance with an embodiment.

FIG. 9 shows an embodiment of a side retention strap comprising a linkage for facilitating anterior movement of the mandibular retainer in accordance with an embodiment.

FIG. 10 and FIG. 11 show the retention strap in further detail in accordance with an embodiment.

FIGS. 12-14 show retainer buttresses for engaging the lateral retention straps in accordance with an embodiment.

FIG. 15 and FIG. 16 illustrate the movement of the retention straps with respect to the buttresses facilitating lateral movement of the retainers.

FIG. 17 and FIG. 18 illustrate anterior movement of the mandibular retainer with respect to the maxilla retainer in accordance with an embodiment.

FIGS. 19-21 illustrate lateral movement of the mandibular retainer with respect to the maxilla retainer.

### DETAILED DESCRIPTION OF THE INVENTION

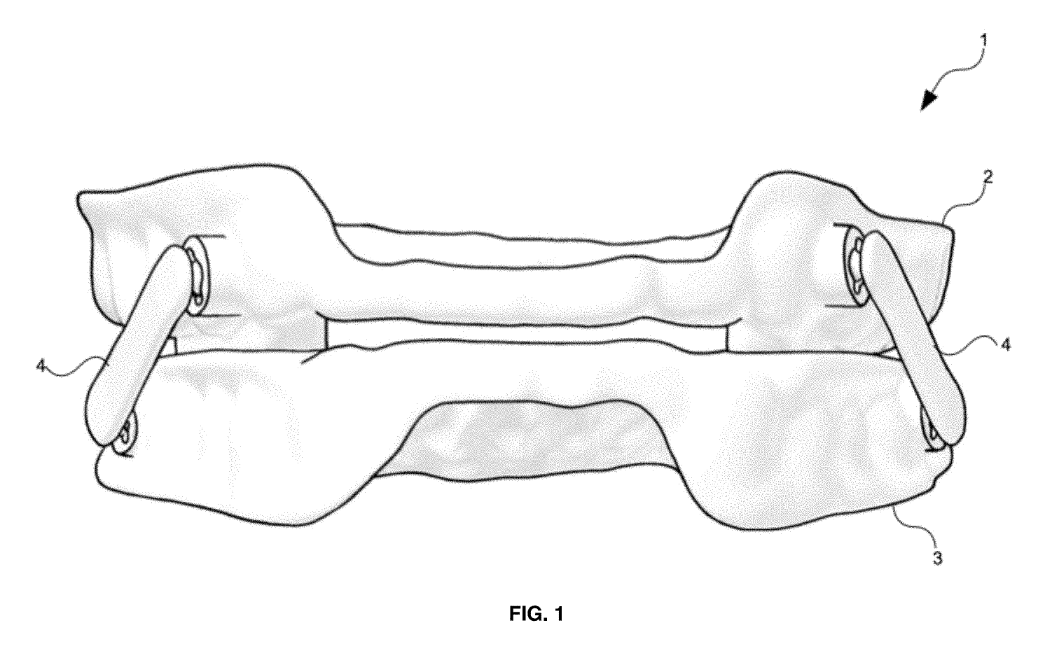

FIG. 1 shows a mandibular advancing intraoral orthosis 1 for addressing snoring and/or sleep apnoea. The orthosis 1 comprises a maxilla retainer 2 and a mandibular retainer 3. The maxilla retainer 2 and mandibular retainers 3 are coupled together utilising a pair of side retention straps 4 such that the mandibular retainer 3 is held in an advanced position with respect to the maxilla retainer 2 in use.

The orthosis 1 is preferably custom fabricated in accordance with intraoral scan data so as to be patient specific, including for teeth fitting and appropriate offsetting of the retainers 2, 3 in the occlusal plane.

The retainers 2, 3 can be manufactured from type 12 polyamide and manufactured utilising selective laser sintering (SLS). The lateral retention straps 4 are preferably also custom fabricated in accordance with the intraoral scan data and furthermore in accordance with a desirous retainer offset in the occlusal plane. The lateral retention straps 4 are non-elastic and, in embodiments, may be manufactured from type 12 polyamide and manufactured utilising selective laser sintering (SLS) or may comprise metal, such as titanium, cobalt chrome or stainless steel.

The retention straps 4 are pivotally coupled to the retainers 2, 3 so as to allow opening of the jaw in use.

As can be appreciated from FIG. 1, the side retention straps 4 couple to the maxilla retainer 2 at an anterior position (such as at a canine region) and to the mandibular retainer 3 at a posterior position (such as at molar region).

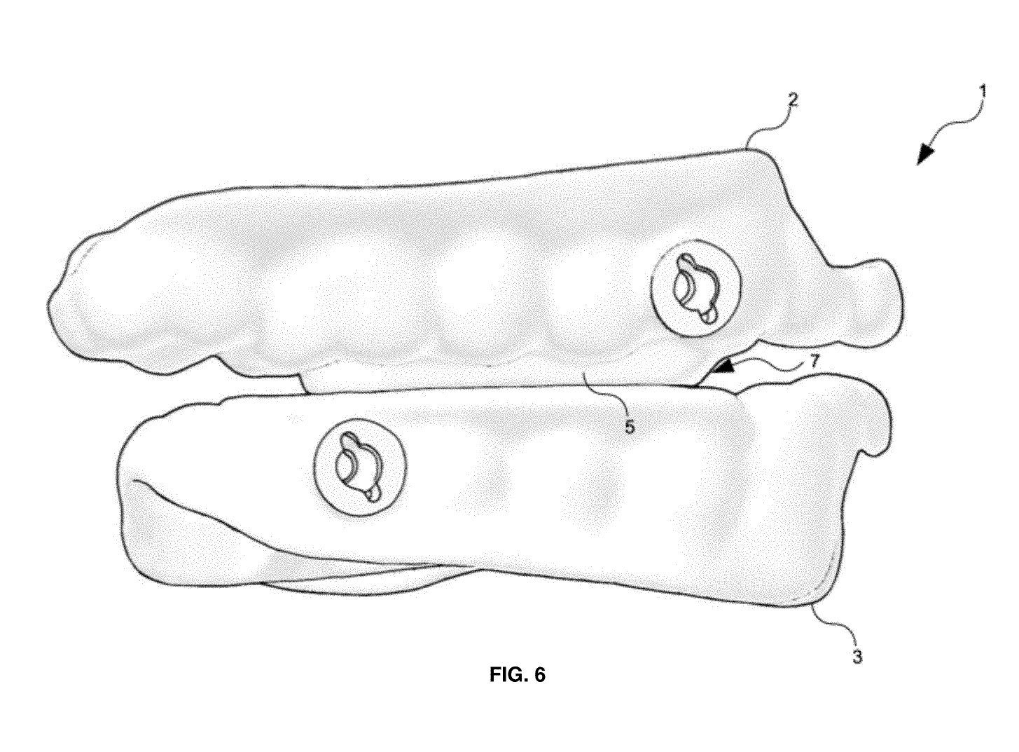

FIG. 7 shows a top plan view of the mandibular retainer 3 and FIG. 8 shows a bottom plan view of the maxilla retainer 2 respectively showing the retainers 2, 3 comprising pairs of planar occlusal faces 5, 6 facilitating the sliding of the retainers 2, 3 across each other.

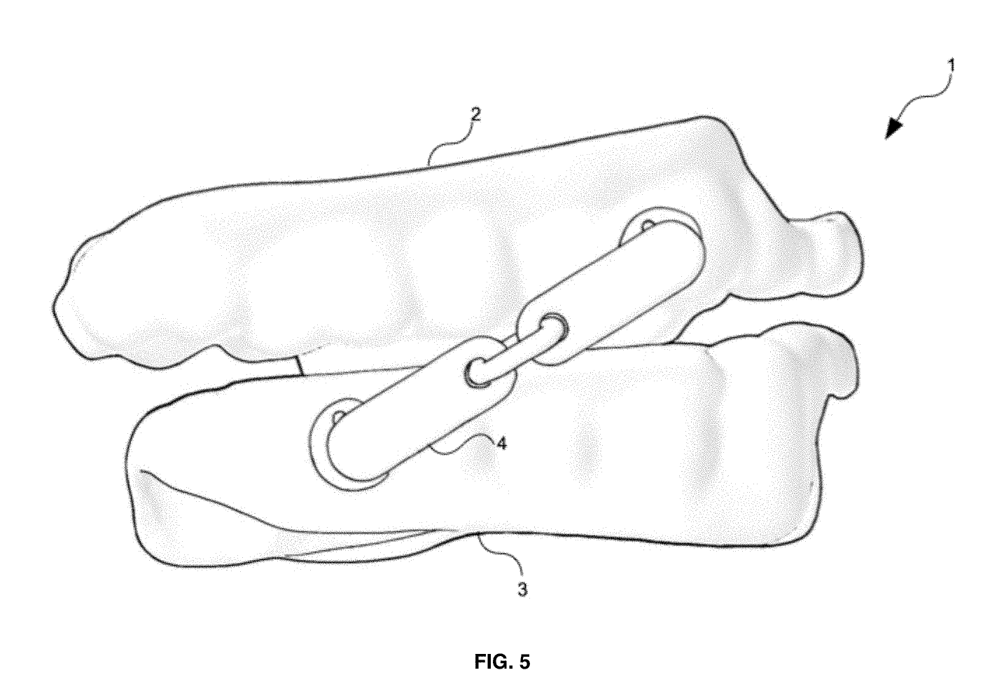

As can be appreciated from FIG. 6, one or both of the occlusal faces may be raised so as to provide an appropriate dentist configured occlusal offset 7 between the retainers 2, 3. The occlusal offset 7 may account for the fact that, when the dentist takes a bite registration for appropriate positioning, such registration may employ an open bite. As such the occlusal faces 5, 6 are made such that the vertical position that the dentist has selected is maintained. As is shown in FIG. 6, it is the maxillary retainer occlusal faces 5 that are raised, however this could be done on the mandibular occlusal faces 6 as well.

Returning to FIGS. 7 and 8, as can be seen, the maxilla retainer occlusal faces 6 may have a smaller surface area as that of the mandibular retainer occlusal faces 5, or vice versa, provided however that contact is maintained across the desirous ranges of motion both laterally, and in embodiments, anteriorly.

As can be appreciated from the above, the occlusal faces 5, 6 allow the sliding of the retainers 2, 3 with respect to each other thereby providing greater freedom of movement during use.

Consideration will now be had for the specific coupling between the side retention straps 4 and the retainers 2, 3 in a manner that facilitates such relative movement.

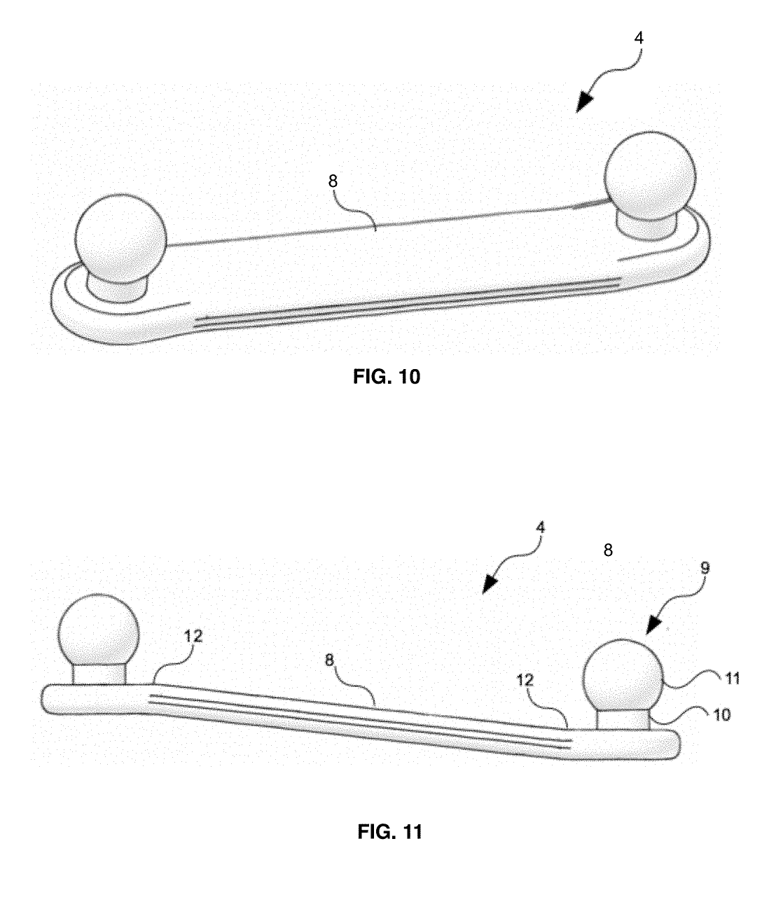

Specifically, turning to FIGS. 10 and 11 there is shown the side retention strap 4 in further detail in accordance with a first embodiment. Specifically, as is shown from the side elevation view of FIG. 11, the side retention strap 4 may comprise an elongate portion 8 which spans between the retainers 2, 3. At opposing distal ends thereof, the retention strap 4 comprises a ball engagement 9 comprising a medial rod 10 being generally perpendicular to the elongate portion 8 and terminating in a medial ball head 11.

As can be appreciated from FIG. 11, the retention strap 4 may be angled at opposing distal junctures 12 so as to allow the anterior ball head 11 to meet the narrower anterior region of the maxilla retainer 2 as compared to the posterior ball head 11 meeting a relatively wider posterior region of the mandibular retainer 3.

FIGS. 12-14 show retainer buttresses 13 for engaging the respective ball engagements 9.

Specifically, as can be appreciated from FIG. 12, the buttress 13 may comprise a generally cylindrical body 14 defining a circular opening 15 into which the respective ball head 11 is pressed for engagement. Opposing edges of the opening 5 may comprise notches 16 for press fit tolerance and to allow for sufficient flexibility (including in the type 12 polyamide) to allow for the ball head 11 to be engaged and disengaged without damage to the orthosis 1 but also provide sufficient retention such that the straps 4 are not displaced during function.

The buttresses 13 may be integrally formed with the respective retainers 2, 3 during manufacture.

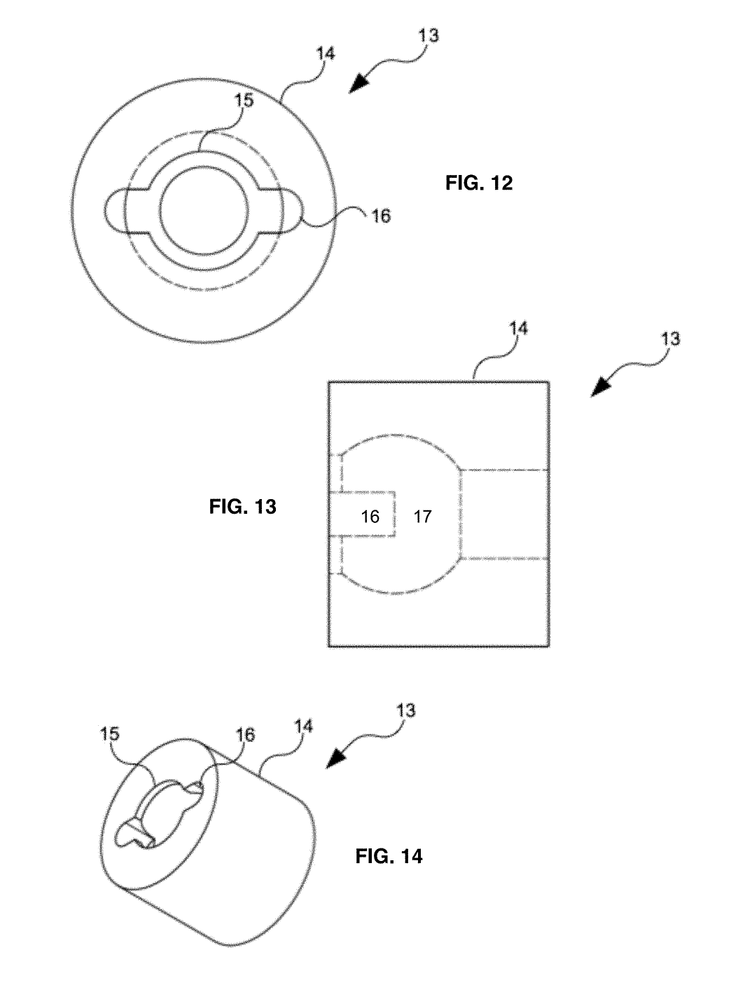

FIG. 13 shows the buttress 13 comprising an interior semi-spherical socket 17 into which the ball head 11 is rotatably coupled. As can be appreciated, such a coupling allows freedom of movement with respect to the transverse plane (so as to facilitate opening and closing of the jaw), freedom of movement with respect to the sagittal plane (so as to facilitate lateral movement of the mandibular retainer 3 with respect to the maxilla retainer 2) and also, in embodiments, with respect to the frontal plane (so as to facilitate anterior movement of the mandibular retainer 3 with respect to the maxilla retainer 2).

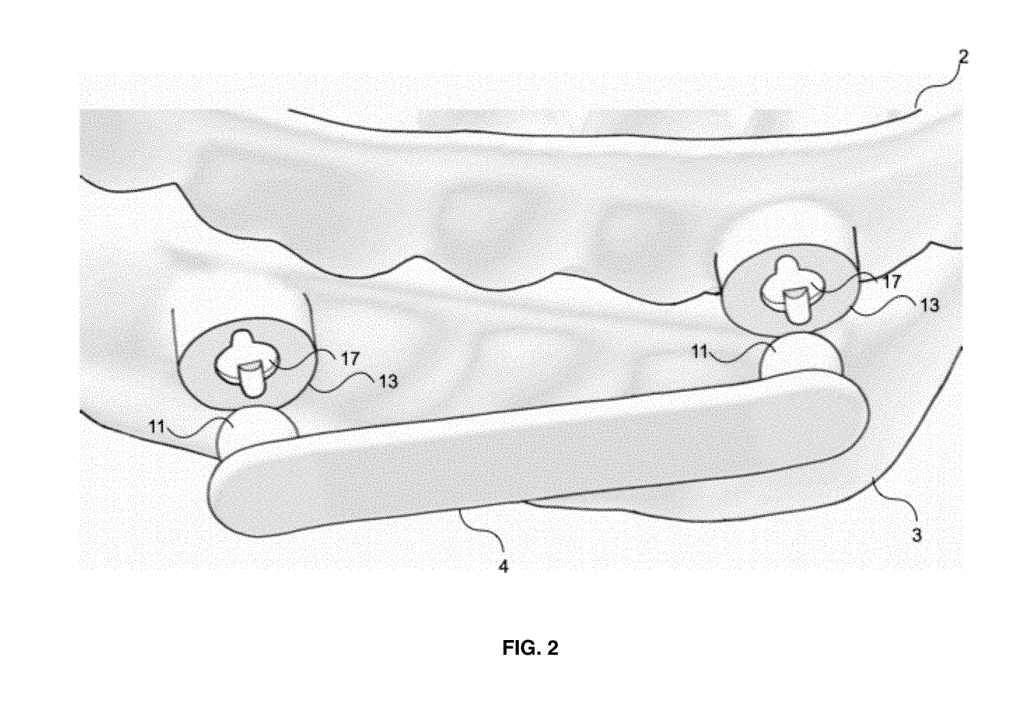

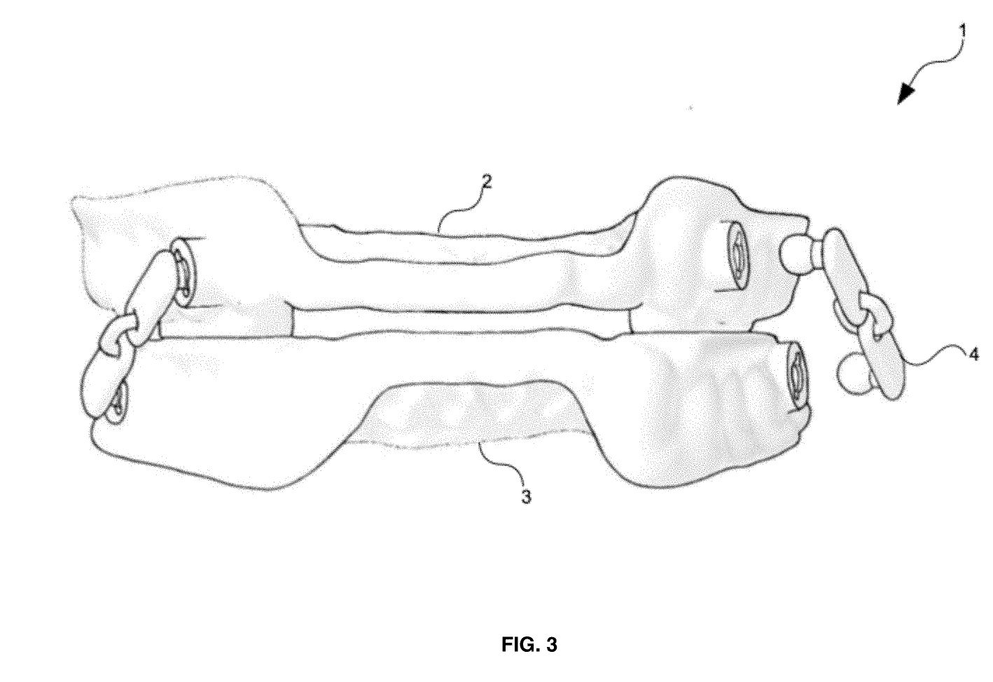

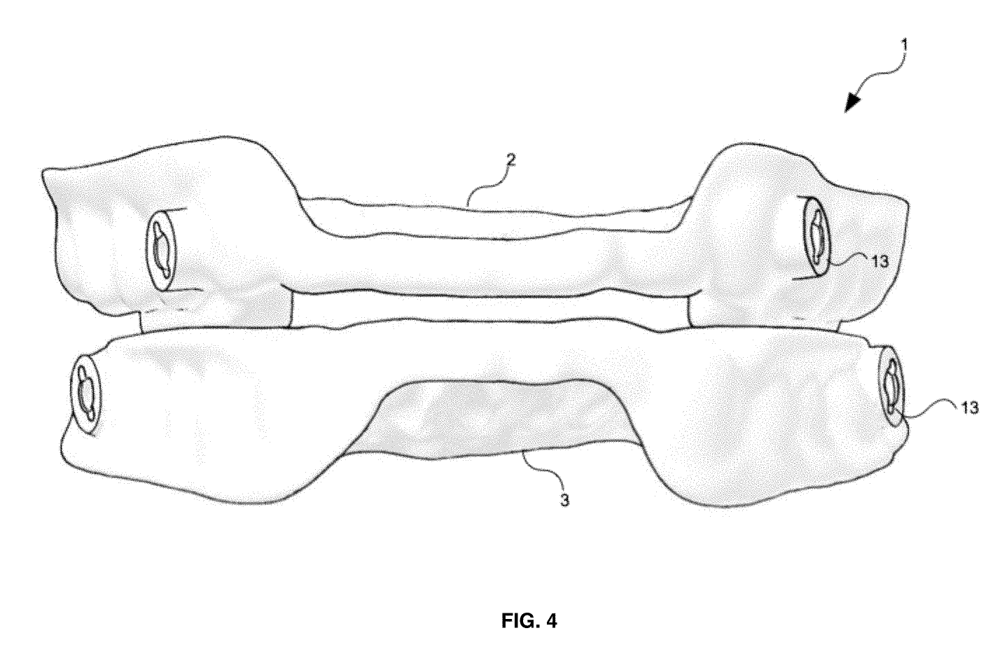

FIG. 2 illustrates the lateral engagement of the retention strap 4 into the respective retainer buttresses 13 prior insertion. As can be appreciated, the coupling allows for convenient press fit insertion wherein each ball head 11 is conveniently pressed into the respective socket 17 of the appropriate buttress 13. As such, the orthosis 1 may be easily disassembled for periodic maintenance or if a strap of different length is required to titrate the mandible to a different position. This would usually be a shorter strap to advance the mandible.

FIG. 1 illustrates the assembled orthosis 1 wherein the retainers 4 have been retained in this manner.

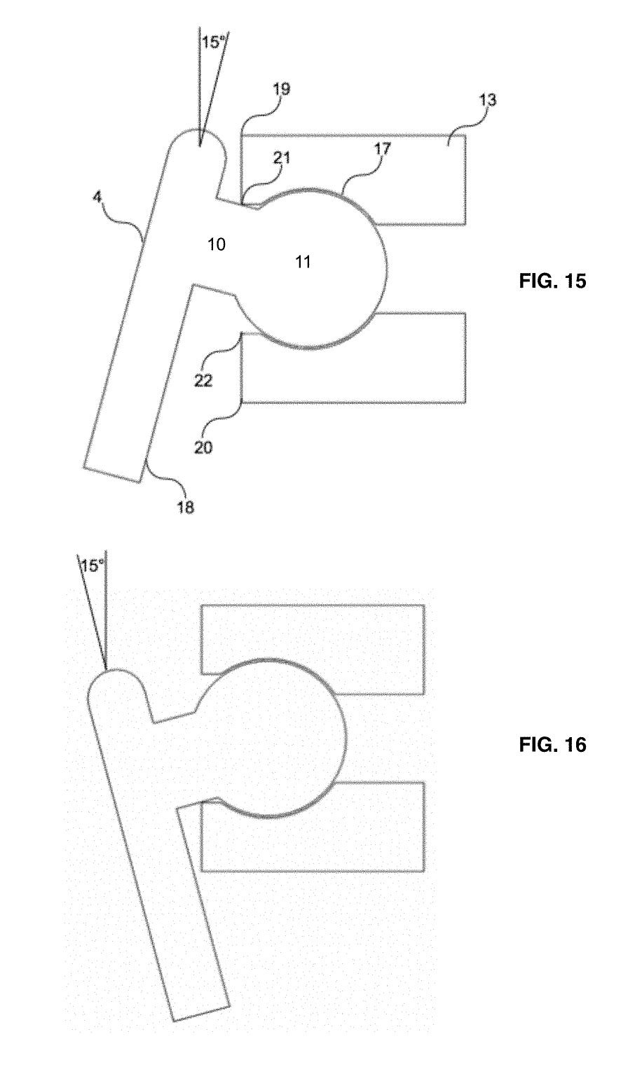

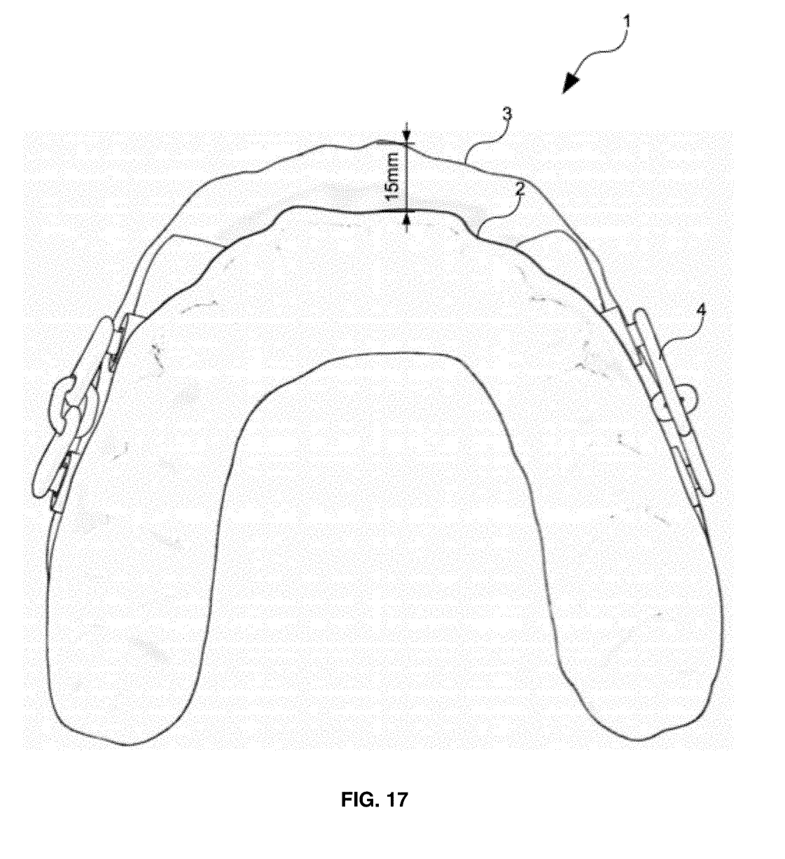

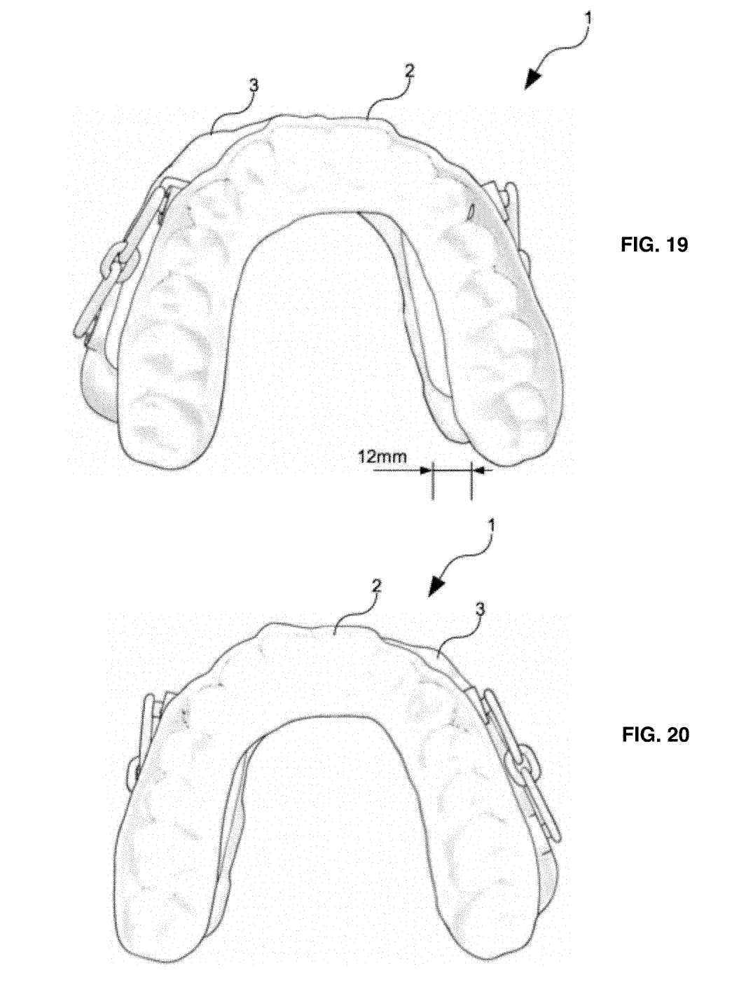

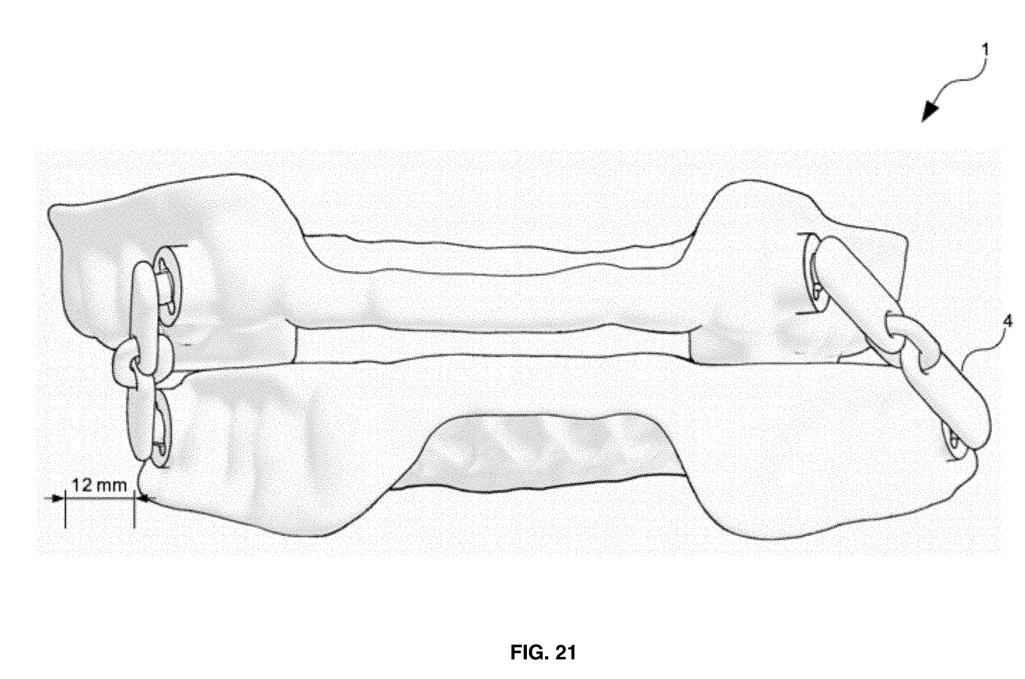

FIGS. 15 and 16 illustrate the lateral movement tolerance conferred by this coupling with respect to the sagittal plane. Specifically, FIG. 15 shows a top plan view of the maxilla retainer buttress 13 having the ball head 11 of the retention strap 4 rotatably coupled within the socket 17. Furthermore, FIG. 15 illustrates the left-handed lateral sliding of the mandibular retainer 3 with respect to the maxilla retainer 2 (corresponding to the example shown in FIG. 19) and FIG. 16 illustrates the right-handed lateral sliding of the mandibular retainer 3 with respect to the maxilla retainer 2 (corresponding to the example shown in FIG. 20).

As can be appreciated, the present coupling tolerates such movement. Specifically, the width of the opening 15 is greater than the diameter of the rod 10 which, in embodiments, may provide lateral movement tolerance with respect to the sagittal plane of about −15° to +15°.

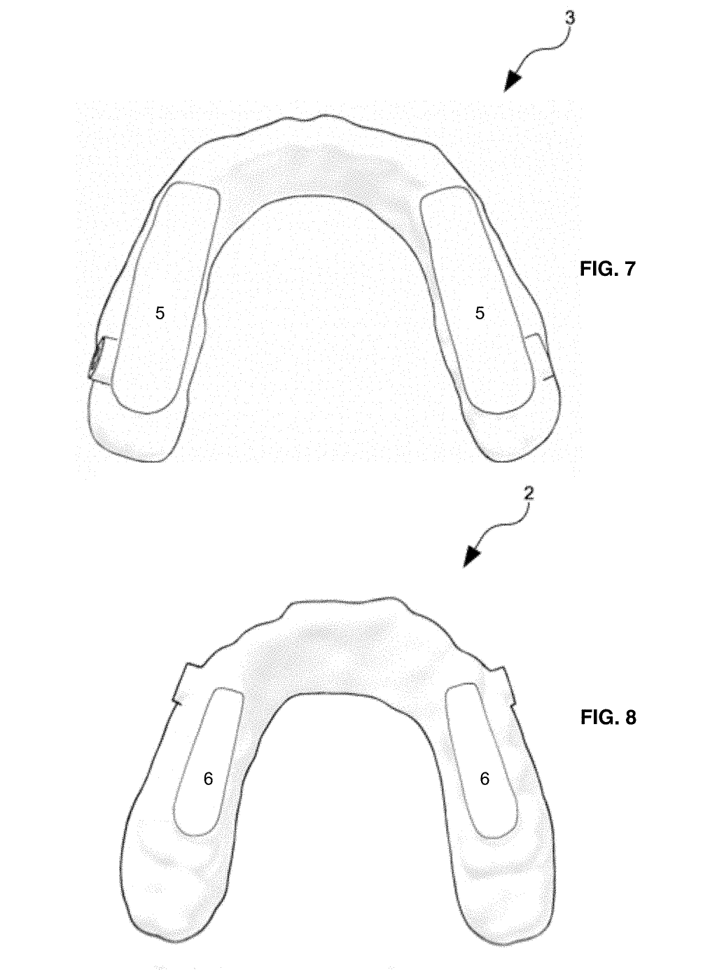

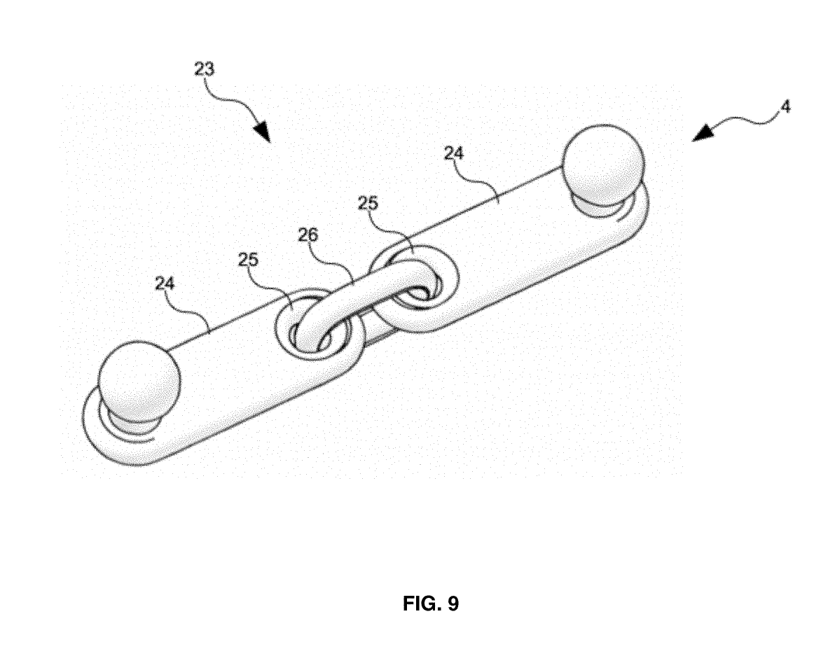

In an embodiment, the retention strap 4 may comprise a linkage as a substantially shown in FIG. 9 further facilitating movement. The linkage 23 also allows the coupling to be placed closer to the teeth and minimise bulk as opposed to a rigid strap that may otherwise contact either the upper or lower retainer in lateral movement, thereby restricting the mandible or potentially dislodging the strap during function. The link 26 in the retention strap 4 allow the strap 4 to bend and resolve such problems.

In embodiments, and as shown in FIGS. 19 and 20, lateral movement tolerance of approximately 12 mm may be provided either way.

Furthermore, as can be appreciated, the length of the rod 10, diameter of the rod 10, and a diameter of the opening 15 and an outer diameter of the buttress body 14 allows such range of motion without the inner faces 18 of the elongate portion 11 pinching against the anterior and posterior edges 19, 20 of the buttress body 14.

In embodiments, the length of each rod 10 allows the inner face to only contact the anterior or posterior edges 19, 20 of the buttress body 14 as the rod 10 contacts the respective anterior or posterior inner edges 21, 22 of the opening 15

As can also be appreciated, the present coupling eliminates or substantially reduces the likelihood of dislocation of the coupling during such lateral movement as the case may be for known orthoses.

Furthermore, as can be appreciated, the present coupling has a low profile allowing the patient the ability to seal the lips normally thereby allowing the retention straps 4 to span between the canine region of the maxilla and the molar region of the mandible.

Such a configuration is advantageous in that it places the retention straps 4 in a more resilient state of tension as opposed to certain prior art orthoses which, on account of being less ergonomic, seek to avoid the maxilla incisor region and therefore place the side retention straps and in opposite configuration and therefore under compression. Such compression may lead to bowing of the retention straps and therefore dislocation or damage. Furthermore orthoses having such a configuration have the maxially coupling in the molar region. This is an area where there are space limitations caused by the Mandibular ramus and may lead to appliances being uncomfortable to use.

FIG. 9 shows the retention strap 4 in accordance with a further embodiment wherein the elongate portion 11 comprises a linkage 23 facilitating anterior movement of the mandibular retainer 3 with respect to the maxilla retainer 2. The linkage 23 allows the retention strap 4 to shorten when compressed while yet having a fixed length under tension.

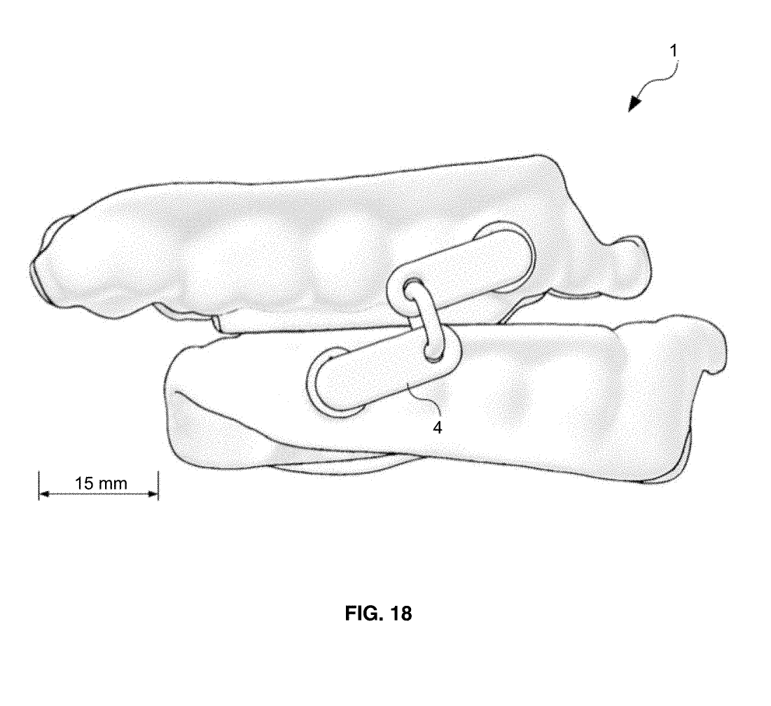

As such, as the mandibular retainer 3 slides forward with respect to the maxilla retainer 2, the linkage 23 allows each retention strap 4 to shorten in the manner substantially shown in FIG. 17 and FIG. 18. As can be seen from FIG. 17 and FIG. 18, the mandibular retainer 3 has moved further anteriorly with respect to the maxilla retainer 2 and wherein, as can be seen, the linkage 23 allows the shortening of each retention strap 4.

In embodiments, as is also represented in FIG. 17 and FIG. 18, such an arrangement may allow for anterior motion of the mandibular retainer 3 by up to approximately 15 mm.

In a specific embodiment as a substantially shown in FIG. 9, the elongate portion 11 of the retention strap 4 is substantially bifurcated comprising distal elongate portions 24, each comprise adjacent profiled apertures 25 held together by a single toroidal-profile link 26. The utilisation of a single link 6 reduces the space requirements of the retention strap 4 and provides a more robust construction as to other retention straps comprising more than one link 26.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

What is claimed is:

1. An intraoral orthosis, comprising:
a maxilla retainer;
a mandibular retainer;
a pair of side retention straps coupling the maxilla retainer to the mandibular retainer, each retention strap spanning between an interior canine region of the maxilla retainer to a posterior molar region of the mandibular retainer and each retention strap comprising an elongate portion wherein:
the maxilla and mandibular retainers comprise respectively abutting pairs of planar maxilla and mandibular retainer occlusal faces facilitating at least lateral sliding of the mandibular retainer against the maxilla retainer; and
the retainers are coupled to the retention straps by couplings at opposing distal ends thereof wherein each coupling comprises:
a lateral retainer buttress comprising an opening to a semi-spherical socket therein; and
a medial retention strap ball engagement, the ball engagement comprising:
a medial rod extending substantially perpendicularly to the elongate portion and terminating in a medial semispherical ball head, the ball head configured for insertion through the buttress opening for engagement and rotatable coupling within the semispherical socket.

2. The intraoral orthosis as claimed in claim 1, wherein the rod comprises an exterior diameter less than that of an interior diameter of the opening.

3. The intraoral orthosis as claimed in claim 1, wherein the rod comprises a rod length and a rod outer diameter and wherein the buttress opening comprises an opening inner diameter and wherein the buttress comprises an buttress outer diameter and wherein the rod length, rod outer diameter, opening inner diameter and buttress outer diameter configured such that an inner face of the elongate portion avoids pinching of respective anterior or posterior edges of the buttress through a lateral range of motion of the mandibular retainer with respect to the maxilla retainer.

4. The intraoral orthosis as claimed in claim 3, wherein the range of motion is approximately −15° to 15° with respect to the sagittal plane.

5. The intraoral orthosis as claimed in claim 1, wherein the retainer buttress is generally cylindrical.

6. The intraoral orthosis as claimed in claim 1, wherein the retainer buttress is integrally formed with a respective retainer.

7. The intraoral orthosis as claimed in claim 1, wherein the elongate portion is angled at a pair of distal junctures such that, in a side elevation view, an anterior distal end is raised with respect to a posterior distal end.

8. The intraoral orthosis as claimed in claim 1, wherein at least one of the pairs of planar maxilla and mandibular retainer occlusal faces is raised so as to provide an occlusal offset between the maxilla retainer and the mandibular retainer.

9. The intraoral orthosis as claimed in claim 1, wherein each retention strap comprises a linkage allowing shortening of the retention strap under compression.

10. The intraoral orthosis as claimed in claim 9, wherein the linkage comprises the elongate portion being bifurcated into two constituent distal elongate portions and wherein adjoining edges of the two distal elongate portions comprise apertures therein through which a link is retained.

11. The intraoral orthosis as claimed in claim 1, wherein the maxilla and mandibular retainers comprise a patient specific teeth fitting inner profile.

12. The intraoral orthosis as claimed in claim 11, wherein the maxilla and mandibular retainers are manufactured from a custom fabrication process using patient specific intraoral scanning data.

13. The intraoral orthosis as claimed in claim 12, wherein the length of the retention straps is custom fabricated in accordance with the intraoral scanning data and a desirous retainer offset along the occlusal plane.

14. The intraoral orthosis as claimed in claim 1, wherein the side retention straps are non-elastic.

15. The intraoral orthosis as claimed in claim 11, wherein the side retention straps comprise type 12 Polyamide.

16. The intraoral orthosis as claimed in claim 14, wherein the side retention straps comprise at least one of titanium, cobalt chrome and stainless steel.
